(19) United States
(12) Patent Application Publication
(10) Pub. No.: US 2006/0134162 A1
(43) Pub. Date: Jun. 22, 2006

(54) Methods for fabricating a drug delivery device

(76) Inventor: Christopher W. Larson, Rochester, NY (US)

Correspondence Address: Bausch & Lomb Incorporated, One Bausch & Lomb Place, Rochester, NY, 14604-2701, US

(21) Appl. No.: 11/013,769

(22) Filed: Dec. 16, 2004

(51) Int. Cl. A61K 6/083; B05D 3/02

(52) U.S. Cl. 424/422; 427/2.26

(57) ABSTRACT

A drug delivery device for placement in the eye includes a drug core comprising a pharmaceutically active agent, and a holder that holds the drug core. The holder is made of a material impermeable to passage of the active agent and includes an opening for passage of the pharmaceutically agent therethrough to eye tissue. The device includes a layer of material permeable to passage of the active agent. This invention provides improved methods that do not require the use of adhesives to form the drug delivery device.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and optionally includes at least one opening for passage of the pharmaceutically agent therethrough to eye tissue. Particularly, this invention provides improved methods of making such devices.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core including a pharmaceutically active agent, and some type of holder for the drug core made of an impermeable material such as silicone or other hydrophobic materials. The holder includes one or more openings for passage of the pharmaceutically active agent through the impermeable material to eye tissue. Many of these devices include at least one layer of material permeable to the active agent, such as polyvinyl alcohol.

Various prior methods of making these types of devices involve the step of using room temperature vulcanizable (RTV) adhesives to adhere the materials from which the device is fabricated, such as the layer of permeable material, after insertion of the drug core in the device. This invention recognized that some adhesions could weaken and deleteriously affect the delivery of the active agent. This invention provides improved methods that do not require the use of adhesives to form the drug delivery device.

### BRIEF DESCRIPTION OF THE DRAWINGS

FIG. 1 is a perspective view of a first embodiment of a drug delivery device of this invention.

FIG. 2 is a cross-sectional view of the device of FIG. 1.

FIG. 3 is a cross-sectional view of the device of FIGS. 1 and 2 during assembly.

FIG. 4 is a cross-sectional view of an embodiment of a drug delivery device.

FIG. 5 is a cross-sectional view of the a second embodiment of a drug delivery device.

### DETAILED DESCRIPTION OF PREFERRED EMBODIMENTS

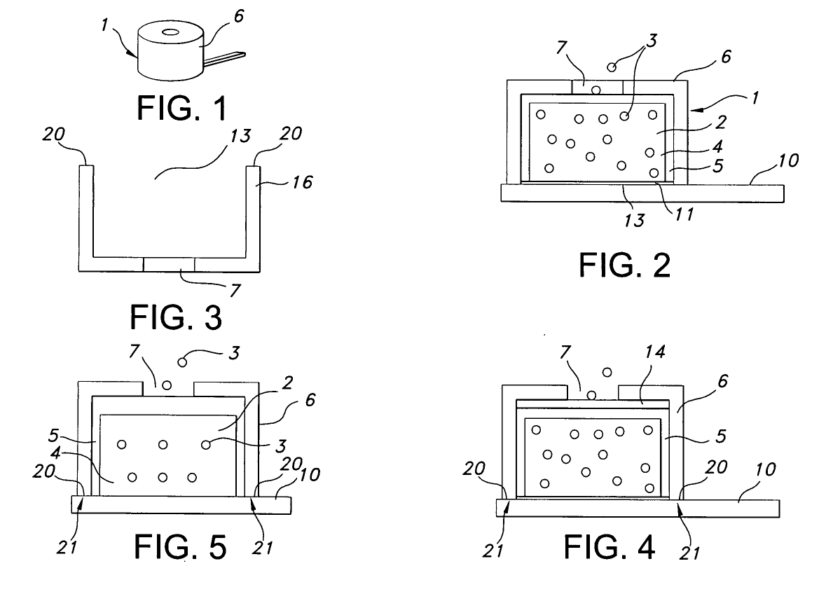

FIG. 1 illustrates a completed device of the invention. FIG. 2 illustrates a prior art device. Device 1 is a sustained release drug delivery device for implanting in the eye. Device 1 includes inner drug core 2 including a pharmaceutically active agent 3.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecarnil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; blood flow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, loteprednol, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

As shown in the illustrated embodiment, active agent 3 may be mixed with a matrix material 4. Preferably, matrix material 4 is a polymeric material that is compatible with body fluids and the eye. Additionally, matrix material should be permeable to passage of the active agent 3 therethrough, particularly when the device is exposed to body fluids. For the illustrated embodiment, the matrix material is PVA. Also, in this embodiment, inner drug core 2 may be coated with a coating 5 of additional matrix material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, at least one passageway 7 may be formed in holder 6 to permit active agent 3 to pass therethrough and contact eye tissue. In other words, active agent passes through any permeable matrix material 4 and permeable coating 5, and exits the device through passageway 7. For the illustrated embodiment, the holder is made of silicone, especially polydimethylsiloxane (PDMS) material. Alternatively, the active agent 3 may be allowed to diffuse through a thin layer of silicone with no passageway 7 (not shown).

A prior method of making a device of the type shown in FIG. 2 includes the following procedures. A cylindrical cup of silicone is separately formed, for example by molding, having a size generally corresponding to the drug core tablet and a shape as generally shown as holder 6 in FIG. 2. This silicone holder is then extracted with a solvent such as isopropanol. Openings 7 are placed in silicone, for example, by boring or with the laser. A drop of liquid PVA is placed into the holder through the open end 13 of the holder, this open end best seen in FIG. 3. Then, the inner drug core tablet is placed into the silicone holder through the same open end 13 and pressed into the cylindrical holder. As a result, the pressing of the tablet causes the liquid PVA to fill the space between the tablet inner core and the silicone holder, thus forming permeable layer 5 shown in FIGS. 1 and 2. For the illustrated embodiment, a layer of adhesive 11 is applied to the open end 13 of the holder to fully enclose the inner drug core tablet at this end. Base 10 is inserted at this end of the device. The liquid PVA and adhesive are cured by heating the assembly.

As mentioned, this invention recognized that some prior methods of forming the device resulted in adhesions that may fail. This invention provides methods of allowing a chemical bond to form between the surfaces to be joined and does not require the use of adhesives to form the drug delivery device.

The surfaces 20 around the open end 13 of the holder 6 and contacting areas 21 of base 10 are subjected to reactive conditions to form sufficient silanol groups on their surfaces to be irreversibly bonded once the surfaces are placed in contact with each other. After the holder 6 and base 10 are in contact, the device assumes the appearance as in FIG. 5.

For the illustrated embodiment, the active agent may be provided in the form of a micronized powder, and then mixed with an aqueous solution of the matrix material, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

An alternate embodiment is illustrated in FIG. 4. In this embodiment, the device further includes a disc 14 made of permeable material covering passageway 7 between the holder 6 and layer 5. For the illustrated embodiment, disc 14 may be preformed from PVA, similar to the material used for layer 5 and matrix material 4. In assembling this embodiment, disc 14 is placed in holder 6 prior to adding the liquid curable material forming layer 5. Additionally, base 10 is elongated. A potential advantage of this embodiment is that the elongation of base 10 allows for its use as a suture tab.

In addition to the illustrated materials, a wide variety of materials may be used to construct the devices of the present invention. The only requirements are that they are inert, non-immunogenic, of the desired permeability and capable of having reactive silanol, hydroxyl, or carboxylic acid groups formed on the surfaces to be bonded. For example, the surface of a silicon-containing polymer, like silicone, will form silanol groups when activated by plasma treatment. Chemical reaction of silanol groups on this activated silicone surface with silanol groups on another activated silicone surface will yield a siloxane bond. This bridging siloxane covalent bond provides a strong mechanical bond when the two surfaces are brought into contact. (Please see Duffy et al., “Rapid Prototyping of Microfluidic Systems in Poly (dimethylsiloxane)” Anal Chem, vol. 70, pp. 4974-4984 (1998)). Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer. When the material used is not capable of having silanol, hydroxyl, or carboxylic acid groups formed on the surfaces to be bonded it may be necessary to apply a coating of a silicone containing material to the surfaces before exposing the surfaces to silanol forming conditions. Typically, the plasma treatment is conducted in a closed chamber at an electric discharge frequency of 13.56 MHz, preferably between about 20 to 500 watts at a pressure of about 0.1 to 1.0 torr, preferably for about 10 seconds to about 10 minutes or more, more preferably about 1 to 10 minutes. Alternatively, plasma treatment can be conducted in a continuous process. Gases such as oxygen, air, argon, nitrogen, and combinations thereof, can be used to provide the reactive silanol groups. Other methods of forming reactive silanol groups include corona and ultraviolet-ozone (UVO) treatment. After treatment, both surfaces to be bonded are brought into contact. Contact times can be a few seconds to several hours. Temperature during contact can be 15° to 70° C., preferably room temperature.

A non-limiting example for forming reactive silanol groups is provided below:

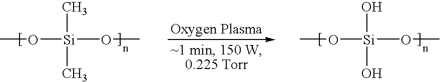

Below is a chemical diagram of the reaction that will take place between the surfaces having silanol groups formed thereon.

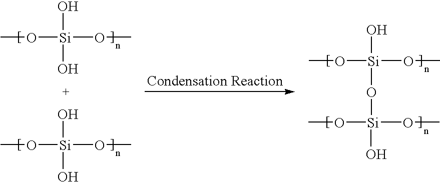

The illustrated embodiment includes a base 10 which may be made of a wide variety of materials, including those mentioned above for the matrix material and/or the holder. Base 10 may be elongated in order to attach the device to a desired location in the eye, for example, by suturing. For the illustrated embodiment, base 10 is made of PVA co-molded with silicone and is adhered to the holder 6 with the reaction of reactive silanol groups. If it is not necessary to suture the device in the eye, base 10 may have a smaller size such that it does not extend substantially beyond holder 6 (see FIG. 5).

According to preferred embodiments, the holder is extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wetability of the holder and improve adherence of the drug core to the holder. Such plasma treatment employs an oxidation plasma in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the plasma treatment is conducted in a closed chamber at an electric discharge frequency of 13.56 MHz, preferably between about 20 to 500 watts at a pressure of about 0.1 to 1.0 torr, preferably for about 10 seconds to about 10 minutes or more, more preferably about 1 to 10 minutes.

The device may be sterilized and packaged. For example, the device may be sterilized by irradiation with gamma radiation.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture base, has a maximum height, width and length each no greater than 10 mm, more preferably no greater than 5 mm, and most preferably no greater than 3 mm.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A method of forming a drug delivery device comprising:
providing a drug holder having at least one surface to be bonded,
providing a base having at least one surface to be bonded,
exposing the surfaces to be bonded to reactive conditions suitable to form reactive groups selected from the group consisting of silanol, hydroxyl, and carboxylic acid groups groups, and
contacting the surfaces to be bonded having the reactive groups with each other under conditions suitable to allow reaction of the reactive groups to occur to form a covalently bonded drug delivery device.

2. The method of claim 1 further comprising loading the drug holder with an active agent before exposing the at least one surface to conditions suitable to form reactive groups.

3. The method of claim 1 further comprising loading the drug holder with an active agent after exposing the at least one surface to conditions suitable to form reactive groups.

4. The method of claim 1 further comprising the step of forming an opening in the holder.

5. The method of claim 1 wherein the step of exposing the at least one surface to be bonded to conditions suitable to form reactive groups comprises exposing the surfaces to be bonded to an oxygen plasma.

6. The method of claim 5 wherein the oxygen plasma is formed at about 20 to 50 watts and about 0.1 to about 1.0 torr.

7. The method of claim 1 wherein the step of exposing the at least one surface to be bonded to conditions suitable to form reactive groups is maintained for a time period from about 10 sec to about 3 min.

8. A drug delivery device formed by the method of claim 2.

9. A drug delivery device formed by the method of claim 3.

10. A drug delivery device formed by the method of claim 4.

11. An assembly comprising:
(a) a medical device implantable in the human eye;
(b) a package for storing the device during storage and shipping; wherein the drug delivery device is the device of claim 8.

12. The assembly of claim 11 wherein the drug delivery device is the device of claim 9.

13. The assembly of claim 11 wherein the drug delivery device is the device of claim 10.

14. The method of claim 1 wherein the reactive groups formed are silanol groups.

15. The method of claim 1 wherein the reactive groups formed are hydroxyl groups.

16. The method of claim 1 wherein the reactive groups formed are carboxylic acid groups.

17. A drug delivery device formed by the method of claim 14.

18. A drug delivery device formed by the method of claim 15.

19. A drug delivery device formed by the method of claim 16.

20. The method of claim 1 wherein the base is elongated to a degree sufficient to provide a suture tab.

21. The device of claim 8 sized and configured for implantation in a human eye.

22. The device of claim 9 sized and configured for implantation in a human eye.

23. The device of claim 10 sized and configured for implantation in a human eye.

24. The device of claim 17 sized and configured for implantation in a human eye.

25. The device of claim 18 sized and configured for implantation in a human eye.

26. The device of claim 19 sized and configured for implantation in a human eye.
